(19) United States
(12) Patent Application Publication
(10) Pub. No.: US 2006/0246033 A1
(43) Pub. Date: Nov. 2, 2006

(54) Injectable bulking agent compositions

(75) Inventor: Lal Ninan, Santa Rosa, CA (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: Cook Biotech Incorporated, West Lafayette, IN (US)

(21) Appl. No.: 11/365,285

(22) Filed: Mar. 1, 2006

Related U.S. Application Data
(60) Provisional application No. 60/657,871, filed on Mar. 2, 2005.

(51) Int. Cl. A61K 38/21; A61K 9/14; A61K 38/18

(52) U.S. Cl. 424/85.5; 424/488; 424/85.6; 424/85.7; 514/12

(57) ABSTRACT

Compositions comprising comminuted extracellular matrix material, a growth factor and a telocollagen are provided. The compositions are useful, for example, as injectable bulking agents. Preferably, the compositions comprise collagen having a telopeptide region.

### RELATED APPLICATIONS

This application claims the benefit of U.S. provisional patent application Ser. No. 60/657,871, filed Mar. 2, 2005, which is incorporated herein by reference in its entirety.

### TECHNICAL FIELD

The present invention relates to compositions that can be employed in injectable bulking agents, as well as kits, methods of manufacture and methods of treatment related to the same.

### BACKGROUND

A variety of bulking agents for percutaneous injection are known that augment, support, or reconfigure anatomic structure. Procedures have been reported in medical literature for correction of various conditions that include the injection of bulking agents within the body.

Many bulking agents comprise collagen. Collagen refers to a group of insoluble fibrous proteins. It is the chief constituent of the fibrils of connective tissue, hide, and tendons. For injection-based treatments, collagens can be emulsified in a pharmaceutical carrier, such as a saline solution, thereby forming an injectable liquid emulsion. The collagen molecule comprises a naturally cross-linked series of multiple triple helical structural units and short non-helical sequences called telopeptides at the end of the triple helical region.

Tissues for use as sources of collagenous bulking agents are typically processed using proteolytic digestion with enzymes such as pepsin to solubilise the naturally cross-linked collagen tissue. Pepsin is the most commonly used enzyme because it is available in pure form from commercial sources and can be employed in an acidic solvent in which the monomer molecules readily dissolve. Although limited proteolysis with pepsin has been extremely useful in preparing relatively large amounts of the various collagens in essentially monomeric form from a number of animal and human tissues, the procedure has limitations. For example, the molecules are obtained with altered nonhelical extremities, and this effectively precludes subsequent studies designed to evaluate the structure and function of the native non-helical regions. Furthermore, since enzyme-solubilised collagen is rich in monomeric collagen but without the species specific to end-peptides, also called telopeptide, collagen fibril reconstruction is greatly inhibited and results in reconstructed fibrils that show low thermal stability as compared with native soluble collagen that includes telopeptides. The use of pepsin in the preparation of injectable collagen-containing compositions also has the practical disadvantage of binding firmly on its substrate and thereby being difficult to remove. Pepsin is a strongly antigenic molecule, and the presence of even a small quantity in an injectible composition can compromise the biocompatibility of the composition.

Another limitation of existing injectable collagen bulking agents, such as collagenic solutions, is subsequent degradation and absorption of the injected agent by normal biological processes following injection. Such processes are commensurate with the metabolism of connective tissue in vivo, and may involve a number of proteolytic enzymes, such as collegenases. The bioabsorption of collagen-containing implants can compromise the long-term effectiveness of injection procedures.

Commercially available injectable collagen products are available as tissue bulking compositions for a variety of applications. Two examples include Zyderm Collagen Implant (ZCI) and Zyplast Collagen Implant (ZI) produced by Collagen Corporation of Palo Alto, Calif. These products are prepared by extracting collagen from cow skin using pepsin digestion. Upon implantation in a patient, however, the volume of injected collagen decreases partly due to the absorption by the body. Furthermore, when injected, the collagen tends to migrate through the tissue; therefore, if specific and local tissue augmentation or bulking is required, such migration would necessitate subsequent injections. Follow up or “top-off” injections at the site are usually necessary with previously developed collagen compositions because the volume decreases. Therefore, volume persistence and shape persistence are desired of an injectable collagen implant. Higher concentrations of collagen help to maintain volume persistence, but are more viscous and therefore more difficult to inject through a delivery needle.

What is needed are compositions useful as improved injectable bulking agents, with minimal absorption or migration of the injection within the body, while retaining ease of extrusion through a delivery needle. Compositions that provide remodelable injectable tissue masses are provided herein, along with kits, and methods of making and using the same. More specifically, the compositions provided herein permits the ingrowth of autologous tissue within the implanted bulking agent mass so as to provide a more permanent mass of bulking tissue at the site of the bulking agent injection.

### SUMMARY

Compositions comprising comminuted extracellular matrix (ECM) material are provided. The compositions are useful, for example, as injectable bulking agents. Preferably, the compositions comprise collagen retaining at least one telopeptide region. The compositions can also comprise one or more growth factors. The growth factors can be naturally present in the material in the compositions, such as the extracellular matrix material, or growth factors can be added to material in the compositions, or both. One preferred source of ECM is porcine SIS. Preferably, the compositions comprise a comminuted ECM material, collagen comprising a telopeptide region, and one or more growth factors. Growth factors can also be added to the compositions, or the level of one or more growth factors in a composition can be increased or decreased.

In a first embodiment, lyophilized particle compositions are provided. Preferably, the composition comprises an extracellular matrix material comprising telocollagen and one or more growth factors. Preferably, a solution of 50 mg of the powder per mL of 0.5 M acetic acid is characterized by a concentration of at least 1.0 ng/mL, or more preferably 10 ng/mL of the first growth factor. An extracellular matrix material can be selected from any suitable source, such as ECM derived from small intestine submucosa (SIS), renal capsule matrix (RCM) or urinary bladder matrix (UBM). Preferably, the extracellular matrix material is porcine SIS. A growth factor can be naturally present in the extracellular matrix material or can be added to the extracellular matrix material. In some embodiments, the extracellular matrix material comprises one or more growth factors. Preferably, the first growth factor is selected from the group consisting of: a fibroblast growth factor, a vascular endothelial growth factor, a platelet derived growth factor, an insulin-like growth factor, a placenta growth factor and a transforming growth factor. In some embodiments, the growth factor(s) are selected from the group consisting of: FGF1, FGF2, FGF3, FGF4, FGF5, FGF6, FGF7, FGF8, FGF9, FGF10, VEGF A, VEGF B, VEGF C, VEGF D, and VEGF E, PIGF, PDGF, EGF, IFN-alpha, IFN-beta, or IFN-gamma, TGF-alpha, and TGF-beta. More preferably, at least one growth factor is FGF-2. In some embodiments, the extracellular matrix material comprises a vascular endothelial growth factor and FGF-2.

In a second embodiment, injectable bulking agents are provided. Preferably, the injectable bulking agent comprises a suspension of particles of an extracellular matrix material in a liquid vehicle, where the extracellular matrix material comprises telocollagen and a first growth factor, and the suspension is characterized by a concentration of at least 1.0 ng/mL of the first growth factor in the suspension. Preferably, the suspension has a concentration of particles of from about 1 mg/mL to about 200 mg/mL of the suspension. Optionally, the suspension can further comprise a gelling agent, a biodegradable polymer, a cryoprotecting agent, a surfactant, a tensoactive agent, or a buffering agent. Preferably, the suspension comprises at least about 2.0 ng/mL of FGF-2. In some embodiments, the injectable bulking agent or lyophilized particles comprise particles having a dimension of between 200 microns and about 600 microns.

In some embodiments, an injectable bulking agent further comprises at least about 90% water by volume or further comprises beta-glucan. In some embodiments, an injectable bulking agent can comprise a phosphate-buffered physiological saline solution containing lidocaine. Compositions disclosed herein as injectable bulking agents or lyophilized powders can optionally further comprise one or more of the following: hyalauronic acid, hyaluronan, sodium hyaluronate, Selenium C. S., Vanadium, Zinc C. S., E-aminocaproic acid or ascorbic acid, a methacrylate polymer, polyvinylpyrrolidone (PVP), or calcium hydroxyapatite (CaHA).

In a third embodiment, methods of manufacturing an injectable bulking agent are provided. Preferably, a method for manufacturing an injectable bulking agent comprises one or more steps of the following steps: (1) combining a comminuted extracellular matrix material material comprising a growth factor with a collagen digestion medium, and (2) maintaining the extracellular matrix material at a pH, temperature and for a duration effective to solubilize a detectable portion of telocollagen in the absence of a proteolytic enzyme. Examples of proteolytic enzymes are pepsin or trypsin. Preferably, the extracellular matrix material is maintained at a pH of less than about 5.0 at a temperature of less than about 10□C. for a duration of at least about 48 hours. A method for manufacturing an injectable bulking agent can further comprise one or more of the following steps: (3) centrifuging the extracellular matrix material to generate a supernatant portion and a pellet portion, (4) isolating the pellet portion, (5) preparing an injectable bulking agent from the pellet portion, (6) forming a suspension by adding a liquid vehicle to the pellet portion, and (7) increasing the pH of the suspension to a physiologically suitable level.

In some embodiments, a liquid vehicle is selected from the group consisting of: a phosphate buffered saline, water, and a physiological solution. Optionally, a method of manufacturing an injectable bulking agent can further comprise the step of adding to the suspension one or more of the following: a gelling agent, a biodegradable polymer, a cryoprotecting agent, a surfactant, a tensoactive agent, and a buffering agent.

Preferably, a method for manufacturing an injectable bulking agent comprises the steps of: combining comminuted SIS comprising telocollagen with a collagen digestion medium, where the collagen digestion medium does not comprise a proteolytic enzyme; maintaining the comminuted SIS and collagen digestion medium together in the absence of a proteolytic enzyme at a pH and temperature and for a time effective to solubilize telocollagen in the SIS; centrifuging the comminuted SIS and collagen digestion medium together to obtain a supernatant portion and a pellet portion; isolating the pellet portion; adding a phosphate buffered saline solution to the pellet portion to form a suspension of SIS particles in the phosphate buffered saline solution; and adjusting the pH of the suspension to a therapeutically effective level. More preferably, the comminuted SIS is maintained at a pH of less than about 5.0, and a temperature of less than 25° C. for at least 48 hours. Most preferably, the comminuted SIS is maintained at a pH of between about 2.0 and 4.0, and a temperature of about 4° C. to about 10° C. for at least 120 hours. Alternatively, the collagen digestion can be performed at a pH of between about 9 and about 11. Subsequently, the pH of the digested suspension is adjusted to a pH of between about 6.0 and 8.0. Preferably, the method for manufacturing the injectable bulking agent further comprises the step of lyophilizing the isolated pellet portion and reconstituting with phosphate buffered saline to form a suspension comprising solid SIS particles at a concentration from about 1 mg/mL to about 200 mg/mL.

In a fourth embodiment, kits comprising the compositions disclosed herein are provided. Preferably, kits can comprise solid particles containing an ECM material with telocollagen and one or more growth factors. Preferably, the solid particles comprise lyophilized ECM particles. In some embodiments, a kit includes a bulking agent composition disclosed in the first embodiment that can be combined with a liquid vehicle to form an injectable bulking agent suspension. For example, a kit can comprise a single-use syringe dose form comprising the injectable bulking agent and a needle. The kit can further comprise a second skin test syringe with the injectable bulking agent, and a topical anesthetic such as lidocane, for assessing a patient's reaction to the bulking agent. In other embodiments, the kit can comprise a lyophilized bulking agent powder or gel and a separately stored liquid vehicle agent. For example, an end user can combine the liquid vehicle agent and the bulking agent to provide an injectable bulking agent formulation. Preferably, the kit comprises a bulking agent comprising a comminuted extracellular matrix material containing telocollagen and at least one growth factor. In one embodiment, the bulking agent comprises a lyophilized bulking agent. The kit can also comprise one or more of the following: a liquid vehicle agent in separate from the lyophilized bulking agent, a single-use implant syringe, a skin test syringe and an implant syringe, or an injection needle.

In a fifth embodiment, various methods of treatment are also provided that include the step of administering a bulking agent composition comprising an ECM material, telocollagen and one or more growth factors. Methods of treatment can comprise injecting a bulking agent comprising the compositions provided herein. Methods of treating sphincter deficiencies are provided, including methods of treating urinary and fecal incontinence and esophageal reflux disorders. Also provided are methods of treating erectile dysfunction, bone fracture, ligament damage, burn, wound or vocal cord impairment, heart tissue defects, and soft tissue defects. Further provided are methods of treating dental conditions and providing tissue augmentation. Some methods of treatment comprise injecting a bulking agent at one or more sites. For example, a bulking agent can be injected at multiple sites symmetrically positioned near an incontinent sphincter muscle.

Preferably, the methods of treatment comprise administering a therapeutically effective amount of a bulking agent comprising a suspension of particles of extracellular matrix material containing telocollagen, the suspension further comprising at least 1 ng/mL of a growth factor, for example by injection. Preferred methods of treating numerous conditions include: (1) methods for treating soft tissue defects, for example comprising the step of increasing the mass of soft tissue by injecting the bulking agent in contact with soft tissue, (2) methods for treating sphincter deficiency (for example, urinary, pyloric or anal sphincter) comprising the localized injection of the bulking agent at one or more sites in or at a therapeutically effective distance from the sphincter muscle to treat conditions such as urinary incontinence, fecal incontinence or esophogeal reflux disease; (3) methods for treating erectile dysfunction comprising localized injection of the bulking agent at one or more sites; (4) methods for treating vocal cords comprising localized injection of the bulking agent at one or more sites near a vocal cord; (5) methods of promoting bone healing or ossification comprising localized injection of the bulking agent at one or more sites near a bone or ligament; and (6) methods of promoting wound or ligament healing comprising localized injection of the bulking agent at one or more sites near a wound or ligament.

In a sixth embodiment, methods for delivering a bioactive agent are provided. Preferably, the method for delivering a bioactive agent comprises administering a therapeutically effective amount of a bulking agent comprising a suspension that includes a bioactive agent and particles of extracellular matrix material containing telocollagen in a liquid vehicle, the suspension further comprising at least 1 ng/mL of a growth factor.

### BRIEF DESCRIPTION OF THE DRAWINGS

FIG. 1A depicts a side view of a tissue structure with an enlarged lumen surrounded by muscle tissue.

FIG. 1B depicts the tissue structure of FIG. 1A immediately after injection of a bulking agent around the enlarged lumen of the tissue from outside a body cavity.

FIG. 1C depicts the tissue structure of FIG. 1A immediately after injection from inside a body cavity of a bulking agent around the enlarged lumen of the tissue.

FIG. 2A is a schematic plan view of an injection needle assembly.

FIG. 2B is a schematic plan view of the injection needle assembly of FIG. 2A with the trocar/obturator assembly being removed.

FIG. 2C is a schematic plan view of the needle assembly of FIG. 2B with a balloon assembly being inserted into the needle assembly;

FIG. 2D is a schematic plan view of the needle assembly of FIG. 2B with a syringe attached to the needle assembly for inflating the balloon;

FIG. 2E is a schematic plan view of the assembly of FIG. 2E with the syringe and balloon assembly being removed;

FIG. 2F is a schematic plan view of the assembly of FIG. 2B with another syringe attached to the needle assembly for injecting a bulking agent into tissue;

FIG. 3 shows components of a single-use injectable bulking agent injection kit;

FIG. 4 illustrates typical injection sites in the dermis for cosmetic and lipodystrophy methods'

FIGS. 5A and 5B illustrates typical injection sites for the treatment of urethral sphincter deficiency;

FIG. 6 illustrates typical injection site for the treatment of lower esophageal sphincter deficiency.

### DETAILED DESCRIPTION OF THE INVENTION

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

The term “effective amount” refers to an amount of an active ingredient sufficient to achieve a desired affect without causing an undesirable side effect. In some cases, it may be necessary to achieve a balance between obtaining a desired effect and limiting the severity of an undesired effect. It will be appreciated that the amount of active ingredient used will vary depending upon the type of active ingredient and the intended use of the composition of the present invention.

The term “about” used with reference to a quantity includes variations in the recited quantity that are equivalent to the quantity recited, for instance an amount that is insubstantially different from a recited quantity for an intended purpose or function.

The term “telocollagen” refers to a collegen molecule retaining the telopeptide region, or the detectable presence of a telopeptide region in any composition described herein as comprising collagen. Preferably, the telopeptide is associated with or bound to a collagen molecule.

The term “collagen” refers to a class of fibrous proteins, largely associated with animal connective tissue. A number of different vertebrate collagens have been identified. There are at least 12 types of collagen. Types I, II and III are the most abundant and form fibrils of similar structure. Type IV collagen forms a two-dimensional reticulum and is a major component of the basal lamina. Collagens are predominantly synthesized by fibroblasts, but epithelial cells also synthesize these proteins. The collagen molecule is built from three peptide chains that are helical in conformation. At the end of the triple helical domain, short non-helical chains, namely telopeptides, having a non-repeating sequence and spanning from 9 to 25 residues, extend beyond the triple helix from both ends of each chain.

The term “liquid vehicle,” refers to any biologically suitable liquid that can be combined with particles to form a suspension, for example an injectable bulking agent suspension comprising particles of SIS with telocollagen suspended in a suitable liquid vehicle. Preferably, liquid vehicles are hydrophilic or water-based solutions such as phosphate-buffered saline (PBS), water, saline, Krebs-Ringer solution containing 5% dextrose, or in any other physiological solution. Alternatively, liquid vehicles can be hydrophobic or amphoteric liquids. Liquid vehicles can also be any suitable polymeric liquid at room temperature (i.e., about 25° C.). Preferably, the suspension is an injectable suspension. Suitable liquid vehicles also include, for example, water, saline, dextrose, glycerol, ethanol, or the like and combinations thereof. In addition, if desired, the vehicle may contain minor amounts of substances such as wetting or emulsifying agents or pH buffering agents.

A “collagen digestion medium” refers a substance that acts to solubilize at least a portion of a telocollagen from the ECM material. Preferably, the collagen digestion medium is an acid that does not oxidize collagen. Nitric acid and sulphuric acid are examples of acids that can oxidize collagen and so are not suitable as collagen digestion media. Preferably, the collagen digestion medium maintains the ECM material at a pH of from about 2 to about 4. More preferably, the collagen digestion medium is acetic acid or hydrochloric acid. Particularly preferred collagen digestion media are 0.50 M acetic acid or 0.01 M hydrochloric acid (HCl). Preferably, the collagen digestion medium does not comprise a proteolytic enzyme such as pepsin. However, a collagen digestion medium that includes a proteolytic enzyme can be used to partially digest an ECM material, for example to lower the concentration of telocollagen in the ECM material. In another embodiment, the collagen digestion medium can have a basic pH, preferably between a pH of about 9 and a pH of about 11.

Abbreviations used in the description are:
ECM: extracellular matrix
EGF: Epidermal growth factor;
Epo: Erythropoietin
IFN: a murine interferon
IGF-I: Insulin-Like Growth Factor-I (also called somatomedin C)
IGF-II: Insulin-Like Growth Factor-II
FGF: any Fibroblast Growth Factor
FGF-1, FGF1, acidic-FGF, or aFGF: acidic Fibroblast

Growth Factor
FGF-2, FGF2, basic-FGF, or bFGF: basic Fibroblast Growth

Factor
FGF-7: Keratinocyte Growth Factor
Fig.: Figure
GF: any Growth Factor
PDGF: Platelet-Derived Growth Factor
PIGF: Placenta Growth Factor
SIS: Small Intestinal Submucosa;
TGF: any Transforming Growth Factor
TGF-alpha or TGF-□: Transforming Growth Factor-alpha
TGF-beta or TGF-□: Transforming Growth Factor-beta
VEGF: any vascular endothelial growth factor
HS: hyaluronic acid
PDGF: platelet derived growth factor
PBS: phosphate buffered saline
   Sources of ECM Material

The compositions provided herein comprise an extracellular matrix (ECM) material can be derived from a variety of suitable sources. Preferably, the ECM material is a remodelable material. The terms “remodelable” or “bioremodelable” refer to the ability of a material to allow or induce host tissue growth, proliferation or regeneration following implantation of the tissue in vivo. Remodeling can occur in various microenvironments within a body, including without limitation soft tissue, a sphincter muscle region, body wall, tendon, ligament, bone and cardiovascular tissues. Upon implantation of a remodelable material, cellular infiltration and neovascularization are typically observed over a period of about 5 days to about 6 months or longer, as the remodelable material acts as a matrix for the ingrowth of adjacent tissue with site-specific structural and functional properties. The remodeling phenomenon which occurs in mammals following implantation of submucosal tissue includes rapid neovascularization and early mononuclear cell accumulation. Mesenchymal and epithelial cell proliferation and differentiation are typically observed by one week after in vivo implantation and extensive deposition of new extracellular matrix occurs almost immediately.

One preferred category of ECM material is submucosal tissue. Submucosal ECM material can be obtained from any suitable source, including without limitation, intestinal submucosa, stomach submucosa, urinary bladder submucosa, and uterine submucosa. Intestinal submucosal tissue is one preferred starting material, and more particularly intestinal submucosa delaminated from both the tunica muscularis and at least the tunica mucosa of warm-blooded vertebrate intestine. More preferably, the ECM material is Tela submucosa, which is a layer of collagen-containing connective tissue occurring under the mucosa in most parts of the alimentary, respiratory, urinary and genital tracts of animals. Examples of suitable ECM materials include renal capsule matrix (RCM), urinary bladder matrix (UBM) and most preferably small intestine submucosa (SIS). Most preferably, the ECM material is obtained from processed intestinal collagen layer derived from the tunic submucosa of porcine small intestine.

“Tela submucosa” refers to a layer of collagen-containing connective tissue occurring under the mucosa in most parts of the alimentary, respiratory, urinary, integumentary, and genital tracts of animals. Tela submucosa, as with many animal tissues, is generally aseptic in its natural state, provided the human or animal does not have an infection or disease. This is particularly the case since the tela submucosa is an internal layer within the alimentary, respiratory, urinary and genital tracts of animals. Accordingly, it is generally not exposed to bacteria and other cellular debris such as the epithelium of the intestinal tract. Preferably, the tela submucosa tissue ECM materials, which are collagen-based and thus predominantly collagen, are derived from the alimentary tract of mammals and most preferably from the intestinal tract of pigs. A most preferred source of whole small intestine is harvested from mature adult pigs weighing greater than about 450 pounds. Intestines harvested from healthy, nondiseased animals will contain blood vessels and blood supply within the intestinal tract, as well as various microbes such as E. coli contained within the lumen of the intestines. Therefore, disinfecting the whole intestine prior to delamination of the tela submucosa substantially removes these contaminants and provides a preferred implantable tela submucosa tissue which is substantially free of blood and blood components as well as any other microbial organisms, pyrogens or other pathogens that may be present. In effect, this procedure is believed to substantially preserve the inherent aseptic state of the tela submucosa, although it should be understood that it is not intended that the present invention be limited by any theory.

Additional information as to submucosa materials useful as ECM materials herein can be found in U.S. Pat. Nos. 4,902,508; 5,554,389; 5,993,844; 6,206,931; 6,099,567; and 6,375,989, as well as published U.S. Patent Applications US2004/0180042A1 and US2004/0137042A1, which are all incorporated herein by reference. For example, the mucosa can also be derived from vertebrate liver tissue as described in WIPO Publication, WO 98/25637, based on PCT application PCT/US97/22727; from gastric mucosa as described in WIPO Publication, WO 98/26291, based on PCT application PCT/US97/22729; from stomach mucosa as described in WIPO Publication, WO98/25636, based on PCT application PCT/US97/23010; or from urinary bladder mucosa as described in U.S. Pat. No. 5,554,389; the disclosures of all are expressly incorporated herein.

Isolation of ECM Material

The ECM material can be isolated from biological tissue by a variety of methods. In general, an ECM material can be obtained from a segment of intestine that is first subjected to abrasion using a longitudinal wiping motion to remove both the outer layers (particularly the tunica serosa and the tunica muscularis) and the inner layers (the luminal portions of the tunica mucosa). Typically the SIS is rinsed with saline and optionally stored in a hydrated or dehydrated state until use as described below. The resulting submucosa tissue typically has a thickness of about 100-200 micrometers, and may consist primarily (greater than 98%) of acellular, eosinophilic staining (H&E stain) ECM material.

Perferably, the source tissue for the ECM material is a tela submucosa that is disinfected prior to delamination by the preparation disclosed in US Patent Application US2004/0180042A1 by Cook et al., published Sep. 16, 2004 and incorporated herein by reference in its entirety. Most preferably, the tunica submucosa of porcine small intestine is processed in this manner to obtain the ECM material. This method is believed to substantially preserve the aseptic state of the tela submucosa layer, particularly if the delamination process occurs under sterile conditions. Specifically, disinfecting the tela submucosa source, followed by removal of a purified matrix including the tela submucosa, e.g. by delaminating the tela submucosa from the tunica muscularis and the tunica mucosa, minimizes the exposure of the tela submucosa to bacteria and other contaminants. In turn, this enables minimizing exposure of the isolated tela submucosa matrix to disinfectants or sterilants if desired, thus substantially preserving the inherent biochemistry of the tela submucosa and many of the tela submucosa's beneficial effects.

Preferably, the ECM material is substantially free of any antibiotics, antiviral agents or any antimicrobial type agents which may affect the inherent biochemistry of the matrix and its efficacy upon implantation. An alternative to the preferred method of ECM material isolation comprises rinsing the delaminated biological tissue in saline and soaking it in an antimicrobial agent, for example as disclosed in U.S. Pat. No. 4,956,178. While such techniques can optionally be practiced to isolate ECM material from submucosa, preferred processes avoid the use of antimicrobial agents and the like which may not only affect the biochemistry of the collagen matrix but also can be unnecessarily introduced into the tissues of the patient.

Other disclosures of methods for the isolation of ECM materials include the preparation of intestinal submucosa described in U.S. Pat. No. 4,902,508, the disclosure of which is incorporated herein by reference. Urinary bladder submucosa and its preparation is described in U.S. Pat. No. 5,554,389, the disclosure of which is incorporated herein by reference.

Digestion of ECM Material

Preferably, the compositions comprise a comminuted ECM material including collagen that has been digested in a manner that preserves at least a detectable portion of the collagen comprising a telopeptide region. Assays for the detection of telocollagen are known in the art, and some are discussed herein. Preferably, the comminuted ECM material is combined with a suitable collagen digestion medium under conditions of pH, temperature and for a duration sufficient to solubilize a desired portion of the telocollagen in the ECM material. More preferably, nearly all the collagen in the digested composition is telocollagen. Most preferably, the comminuted ECM material is digested without contacting a proteolytic enzyme, such as trypsin or pepsin.

The isolated ECM material is digested to solubilize telocollagen in any suitable manner that permits at least a portion of the collagen molecules therein to retain their telopeptide region(s), and does not completely impair any remodelable properties of the ECM material. Preferably, the ECM material processing conditions are also selected to maintain a desired level of growth factors with the telocollagen. Any suitable growth factor can be naturally present in, or added to, the ECM material. The isolated ECM material is processed by comminution and by maintaining the ECM material at a pH, temperature and for a time sufficient to solubilize telocollagen from the ECM material. Preferably, the isolated ECM material is digested by combining the comminuted ECM material with a collagen digestion medium.

Preferably, collagen is solubilized from the isolated ECM material without removing the telopeptide region. Exposure to a proteolytic enzyme is believed to remove the telopeptide portion of collagen in an isolated ECM material. However, some embodiments provide partial digestion of the isolated ECM material that is exposed to a proteolytic enzyme under conditions that limit the action of the proteolytic enzyme on the ECM material so as to preserve some portion of the collagen as telocollagen in the processed ECM material.

The compositions are prepared as solutions or suspensions of ECM material, such as intestinal submucosa, by comminuting the isolated ECM material and combining the comminuted ECM material with a suitable collagen digestion medium. The isolated ECM material can be comminuted by any suitable method, including tearing, cutting, grinding, shearing and the like. In one embodiment, grinding a submucosa ECM material can be performed in a frozen or freeze-dried state although good results can be obtained as well by subjecting a suspension of pieces of the submucosa to treatment in a high speed (high shear) blender and dewatering, if necessary, by centrifuging and decanting excess water.

Preferably, the temperature of the ECM material during processing is maintained below that at which collagen converts to gelatin. Preferably, the temperature should be maintained below 40° C., more preferably below about 25° C., more preferably below about 10° C., and most preferably at about 4° C. or lower. The ECM material can optionally be subjected to some form of agitation during the process described above.

In some embodiments, comminuted ECM material is maintained at an acidic pH that is effective to solubilize telocollagen at a given temperature. Some embodiments provide for maintaining comminuted ECM material at a pH of less than 6.0, including at a pH's of 5.5, 5.0, 4.8, 4.6, 4.4, 4.2, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, or lower. Preferably, the pH is maintained at a pH of about 4.0 or lower, most preferably between a pH of about 2.0 and 4.0. In some embodiments, the comminuted material is contacted with a suitable acid to solubilize the telocollagen. A suitable acid is any acid and associated concentration that is non-oxidizing to the collagen in the ECM material. Given these parameters, the suitable acids and associated acid concentrations can be selected by one skilled in the art. In some embodiments, the acid is selected from the group consisting of ascorbic acid, acetic acid, acetyl salicylic acid, benzoic acid, citric acid, glutamic acid, glycolic acid, lactic acid, malic acid, salicylic acid, and hydrochloric acid. Particularly preferred acids and acid concentrations include 0.5 M acetic acid and 0.01 M hydrochloric acid (HCl). However, certain concentrations of oxidizing acids such as nitric or sulfuric acids may not be suitable. In some embodiments, the pH of a comminuted ECM solution can be adjusted from time to time. Typically a strong acid such as hydrochloric acid is added. For example, in one embodiment, a 1.0N hydrochloric acid solution may be added in small amounts to adjust the pH to around 3.5.

In preferred embodiments, the comminuted ECM material is not exposed to a proteolytic enzyme so as to maximize the levels of solubilized telocollagen. However, in other embodiments, the comminuted ECM material is partially digested by a proteolytic enzyme such as trypsin or pepsin. The pH can be adjusted to best suit the level of enzymatic activity desired in embodiments where a proteolytic enzyme is employed, as understood in the art. For example, a pH of about 2.0 may be optimal for partial digestion with pepsin. Partial digestion may increase the rate of collagen solubilization while decreasing the percentage of solubilized telocollagen.

In another embodiment, the comminuted ECM material is combined with a basic collagen digestion medium to solubilize at least a portion of the telocollagen. Some embodiments provide for maintaining comminuted ECM material at a pH of about 9.0 or greater, including at a pH's of 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, or higher. Preferably, the pH is maintained at a pH of about 9.0 or higher, most preferably between a pH of about 9.0 and 11.0. In some embodiments, the comminuted material is contacted with a suitable base to solubilize the telocollagen. The pH can be raised in any way that solubilizes a desired amount of telocollagen, for example by addition of sodium hydroxide (NaOH) solution or any suitable base. The suitable bases and associated base concentrations can be selected by one skilled in the art.

The pH and a temperature of the comminuted ECM material in the collagen digestion medium are selected and maintained long enough to solubilize a desired amount of collagen from the ECM material. In some embodiments, the pH, temperature and duration of these conditions are selected to preserve growth factors in the ECM material used to form the compositions, particularly when an SIS ECM material is used. Determining the optimal temperature, pH and time can be determined by one of skill in the art. In one embodiment, comminuted ECM material is maintained in the absence of a proteolytic enzyme at an acidic pH of between about 2.0 and 4.0 and a temperature of between about 4° C. and 10° C. for a period of at least about 48 hours, preferably more than 72 hours, more preferably more than 96 hours and most preferably more than 120 hours.

Preferably, the telocollagen in the compositions disclosed herein comprises collagen molecules retaining one ore more telopeptide regions. The compositions prepared from the pellet portion of the ECM material after processing and centrifugation are believed to retain the telopeptide region to a greater extent than the collagen in the supernatant region. Therefore, compositions disclosed herein preferably are prepared from the pellet portion. Typically, only a portion of the collagen will be solubilized from the ECM material by the time selected for maintaining conditions of pH and temperature of the ECM material. The solubilized portion of collagen can be separated by spinning the ECM material containing the solubulized colagen in a centrifuge, resulting in precipitation of the non-solubilized telocollagen in the pellet and the solubilized supernatant in the supernatant. However, either the pellet portion or the supernatant portion can be isolated and be formulated to an injectable bulking agent in various embodiments. In one embodiment, the pellet portion is isolated, the pH adjusted to a therapeutically effective level, and the pellet portion is formulated as an injectable bulking agent. For example, formulation of the pellet portion can include the step of lyophilizing the pellet portion and reconsitituting the lyophilized pellet portion in a suitable liquid agent, such as PBS. The pellet portion can comprise any suitable

In some embodiments, the comminuted ECM material, preferably SIS, can be dried to form a powder, for example a lyophilized powder. Thereafter, it can be hydrated, that is, combined with a suitable liquid vehicle such as water or buffered saline, and optionally other pharmaceutically acceptable excipients, to form a composition as a fluid having a viscosity that is preferably between about 2 to about 300,000 cps at 25° C.

The ECM material can be used alone, or in combination with one or more additional bioactive agents such as physiologically compatible minerals, growth factors, antibiotics, chemotherapeutic agents, antigen, antibodies, enzymes, and hormones.

Detecting Telocollagen

Any suitable assay known in the art can be used to identify the presence of telocollagen in a composition or bulking agent disclosed herein. A telocollagen can be identified by the detection of telocollagen or by detection of a telopeptide by at least one assay, including but not limited to those listed below. Assays can be used to detect the presence of telocollagen by either detecting the presence of collagen molecules comprising one or more attached telopeptide regions or by detecting telopeptides in a pellet or supernatant portion of the partially digested ECM material and correlating a lower level of free (unattached) telopeptide to a higher amount of telocollagen that includes the telopeptide. Preferably, a sample comprising telocollagen is obtained by preparing a collagen composition with limited or no exposure to a proteolytic enzyme, under conditions that allow at least a portion of the collagen molecules in the collagen composition to retain at least one telopeptide region. Most preferably, the collagen composition is prepared without contacting a proteolytic enzyme such as pepsin.

One assay is a quantitative immunoassay for the cross-linked carboxyterminal telopeptide of human type I collagen disclosed by Risteli J, et al., “Radioimmunoassay for the pyridinoline cross-linked carboxy-terminal telopeptide of type I collagen: a new serum marker of bone collagen degradation,” Clin Chem. April 1993;39(4):635-40. Briefly, 100 L samples (tissue extracts, diluted enzyme digests or serum samples) can be incubated for 2 h at 37° C. with 200 L of iodinated tracer and 200 L of an antiserum diluted in 0.5% normal rabbit serum to bind 50% of the tracer. Then 500 L of a second antibody-polyethylene glycol (PEG) suspension (20 mL of goat antirabbit immunoglobulin antiserum and 150 g of PEG (MW 6000) in 1 L of phosphate-buffered saline (PBS) containing 0.04% Tween-20 can be added and vortex-mixed. After 30 min at room temperature, the bound fraction can be separated by centrifugation (2000×g, 30 min, at 4° C.). The supernatant containing the unbound tracer can be decanted and the radioactivity in the precipitate counted with a 1470 Wizard' gamma counter. The samples can be diluted in PBS-Tween. The intra-assay variation for ICTP is between 2.8-6.2% and inter-assay variation between 4.1-7.9%. The HHL-cross-linked telopeptide variant can be assayed essentially similarly to ICTP by an in-house method using a synthetic peptide, SP4 (SAGFDFSFLPQPPQEKY; produced by Neosystem Laboratories, Strasbourg, France), derived from the carboxyterminal telopeptide region of type I collagen as a tracer and standard antigen. The antiserum used can be produced in a rabbit against the divalently cross-linked carboxy-terminal telopeptide antigen of human type I collagen.

Other examples of assays that are suitable for detection of telocolagen in a composition or bulking agent include the following assays, which are incorporated herein by reference: Montagnani A et al., “A new serum assay to measure N-terminal fragment of telopeptide of type I collagen in patients with renal osteodystrophy,” Eur. J. Intern. Med. May 2003;14(3):172-177; Lung F D, et al., “Binding potency of peptide fragments of type 1 collagen cross-linked N-telopeptide measured by an enzyme-linked immunosorbant assay,” Protein Pept Left. October 2002;9(5):451-7; Garnero P, et al., “Evaluation of a fully automated serum assay for C-terminal cross-linking telopeptide of type I collagen in osteoporosis,” Clin. Chem. April 2001;47(4):694-702; and Pagani F, et al., “Evaluation of a fully automated assay to measure C-telopeptide of type I collagen in serum,” Clin Chem Lab Med. November 2000;38(11):1111-3. The OSTEOMARK® immunoassay for detection of cross-linked N-telopeptides of type I collagen (NTx) provides a monoclonal antibody employed in the 96-well enzyme-linked immunosorbant assay (ELISA) specifically that binds the alpha(I) telopeptide chain of type I collagen QYDGKGVG.

Preferably, injectable compositions are biocompatible. “Biocompatibility” refers to the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests assay as to a material's toxicity, infectivity, pyrogenicity, irritation potential, reactivity, hemolytic activity, carcinogenicity, and/or immunogenicity. A biocompatible structure or material when introduced into a majority of patients will not cause an adverse reaction or response. In addition, it is contemplated that biocompatibility can be effected by other contaminants such as prions, surfactants, oligonucleotides, and other biocompatibility effecting agents or contaminants.

Growth Factors

The compositions provided herein preferably contain one or more growth factors. Growth factors in a composition may be, for example, naturally present in and retained by an ECM material such as SIS (endogenous growth factors). Naturally occurring levels of growth factors in an ECM material may optionally be augmented or diminished. Growth factors may also be added to an ECM material or bulking agent composition (exogenous growth factors). Growth factors may also be separately added to a composition, such as a bulking agent composition. Any growth factors that will not significantly diminish the intended function of the composition, for example as an injectable bulking agent, can be included in a composition. Growth factors that promote post-injection retention and remodeling of compositions used in injectable bulking agents are particularly preferred.

In some embodiments, the compositions comprise an ECM material that itself comprises one or more growth factors. For example, submucosa or other ECM materials may include one or more growth factors. Without being bound to theory, it is believed that the presence of one or more growth factors may promote remodeling of the injectable bulking agent, resulting in tissue regrowth before diminution of the injected agent by biodegradation of the telocollagen.

ECM materials having concentrations of 1 ng/mL of one or more growth factors are particularly preferred. Non-limiting examples of growth factors that can be included in compositions useful in injectable bulking agents include: fibroblast growth factors (FGF) (e.g., FGF1, FGF2, FGF3, FGF4, FGF5, FGF6, FGF7, FGF8, FGF9, and FGF10), epidermal growth factor, keratinocyte growth factor, vascular endothelial cell growth factors (VEGF) (e.g., VEGF A, B, C, D, and E), placenta growth factor (PIGF), platelet-derived growth factor (PDGF), epidermal growth factor (EGF), interferons (IFN) (e.g., IFN-alpha, beta, or gamma), transforming growth factors (TGF) (e.g., TGF.alpha or beta), tumor necrosis factor-.alpha, an interleukin (IL) (e.g., IL-1-IL-18), Osterix (See, e.g., Tai G. et al., “Differentiation of osteoblasts from murine embryonic stem cells by overexpression of the transcriptional factor osterix,“Tissue Eng. September-October; 10(9-10):1456-66, incorporated herein by reference in its entirety), Hedgehogs (e.g., sonic or desert) (See, e.g., Adolphe C. et al., “An in vivo comparative study of sonic, desert and Indian hedgehog reveals that hedgehog pathway activity regulates epidermal stem cell homeostasis,” Development. October 2004;131(20):5009-19. Epub Sep. 15, 2004 incorporated herein by reference in its entirety), bone morphogenic proteins, basic fibroblast growth factor (bFGF), parathyroid hormone, calcitonin prostaglandins, ascorbic acid, and hepatocyte growth factor. In one embodiment, a composition comprises at least one fibroblast growth factor, preferably basic fibroblast growth factor FGF-2. In some embodiments, a composition comprises at least one Transforming Growth Factor, preferably TGF-beta. In one preferred embodiment, a composition comprises both FGF-2 and TGF-beta. Other preferred growth factors include one or more types of EGFs (epidermal growth factors), PDGFs (platelet derived growth factors), and VEGFs (vascular endothelial growth factor). See, e.g., Sachiyo Ogawa, et al., “A Novel Type of Vascular Endothelial Growth Factor, VEGF-E (NZ-7 VEGF), Preferentially Utilizes KDR/Flk-1 Receptor and Carries a Potent Mitotic Activity without Heparin-binding Domain,” J Biol Chem, Vol. 273, Issue 47, 31273-31282, Nov. 20, 1998, incorporated herein by reference). Other examples of growth factors include: Brain-derived Neurotrophic Factor, Epidermal Growth Factor, Fibroblast Growth Factor, Endothelial cell growth supplement, Granulocyte-Macrophage Colony-Stimulating Factor, Hepatocyte Growth Factor, Insulin-like Growth Factor, Interleukins, Leukemia Inhibitory Factor, Nerve Growth Factor, Platelet-Derived Growth Factor, Transforming Growth Factor, Tumor Necrosis Factor, and Vascular Endothelial Growth Factor.

The following non-limiting examples of other references relating to growth factors and ECM materials are incorporated herein by reference: Zheng B, Clemmons DR, “Methods for preparing extracellular matrix and quantifying insulin-like growth factor-binding protein binding to the ECM,” Methods Mol Biol. 2000;139:221-30; Rosso F, et. al., “From cell-ECM interactions to tissue engineering,” J Cell Physiol. May 2004;199(2):174-80; Pollak Minn., “Insulin-like growth factors and neoplasia,” Novartis Found Symp. 2004;262:84-98; discussion 98-107, 265-8; Liu X, et al., “Synergetic effect of interleukin-4 and transforming growth factor-beta1 on type I collagen gel contraction and degradation by HFL-1 cells: implication in tissue remodeling,” Chest. March 2003;123(3 Suppl):427S-8S and Shukla A, et al., “Perspective article: transforming growth factor-beta: crossroad of glucocorticoid and bleomycin regulation of collagen synthesis in lung fibroblasts,” Wound Repair Regen. May-June 1999;7(3):133-40.

Preferably, an injectable composition comprises solid particles having 1 ng/mL or higher levels of FGF-1 or FGF-2. Acidic fibroblast growth factor (aFGF), also referred to as FGF-1, is a monomeric, acidic protein of approximately 18 kDa. It shares about 55% homology with the basic protein FGF-2. Basic fibroblast growth factor (bFGF), also referred to as FGF-2, is a 16.5 Kd 146 amino acid protein that belongs to the FGF family, which now comprises more than 22 structurally related polypeptides. One of the key differences between the various FGFs is the presence or absence of the leader sequence required for conventional peptide secretion (absent in FGF-1 and FGF-2). Another difference is the varied affinity for the different isoforms of FGF receptors. As for most heparin-binding growth factors, bFGF binds with high affinity to cellular heparin sulfates and, with even higher affinity, to its own tyrosine kinase receptors (FGF receptors 1 and 2). The ability of bFGF to bind cell surface and matrix heparin sulfates serves both to prolong its effective tissue half-life and to facilitate its binding to the high affinity receptors.

More preferably, the injectable composition comprises at least 1, 10, 100 or 1000 ng of FGF-2 per mL of solution. FGF-2 is a pluripotent mitogen believed to be capable of stimulating migration and proliferation of a variety of cell types including fibroblasts, macrophages, smooth muscle and endothelial cells. In addition to these mitogenic properties, FGF-2 is believed to stimulate endothelial production of various proteases, including plasminogen activator and matrix metalloproteinases, induce significant vasodilation through stimulation of nitric oxide release and promote chemotaxis. FGF-2 binds avidly (Kd 10⁻⁹ M) to endothelial cell surface heparin sulfates. This interaction serves to prolong effective tissue half-life of the FGF-2 protein, facilitates its binding to its high-affinity receptors and plays a key role in stimulation of cell proliferation and migration. FGF-2 also possesses a plethora of other biological effects such as the ability to stimulate NO release, to synthesize various proteases, including plasminogen activator and matrix metalloproteinases, and to induce chemotaxis. Homozygous deletion of the bFGF gene is associated with decreased vascular smooth muscle contractility, low blood pressure and thrombocytosis.

The concentration of FGF-2 in a composition comprising an extracellular matrix material can be detected using any suitable assay. Preferably, the assay selected provides a minimum detectable dose (MDD) of FGF basic of at least 1 pg/mL solution. More preferably, the MDD of an assay is at least 0.5 pg/mL FGF basic in solution.

A preferred method for detection of FGF-2 in a composition is the QUANTIKINE HS® Human FGF basic Immunoassay. The QUANTIKINE HS® FGF basic Immunoassay kit is a 6.5 hour solid phase ELISA designed to measure FGF basic levels in serum, plasma, and urine. The QUANTIKINE HS® FGF basic Immunoassay contains E. coli-expressed recombinant human FGF basic and antibodies raised against the recombinant factor. It has been shown to quantitate recombinant human FGF basic accurately. Results obtained using natural human FGF basic showed linear curves that were parallel to the standard curves obtained using the Quantikine HS kit standards. These results indicate that the QUANTIKINE HS® FGF basic Immunoassay kit can be used to determine relative mass values for natural FGF-2.

The QUANTIKINE HS® Human FGF basic Immunoassay employs a quantitative sandwich enzyme immunoassay technique. Briefly, a monoclonal antibody specific for FGF basic is pre-coated onto a microplate. Standards and samples are pipetted into the wells and any FGF basic present is bound by the immobilized antibody. After washing away any unbound substances, an enzyme-linked monoclonal antibody specific for FGF basic is added to the wells. Following a wash to remove any unbound antibody-enzyme reagent, a substrate solution is added to the wells. After an incubation period, an amplifier solution is added to the wells and color develops in proportion to the amount of FGF basic bound in the initial step. The color development is stopped and the intensity of the color is measured.

Optionally, the FGF-2 detection assay further comprises an amplification system. The QUANTIKINE HS® Immunoassay kit uses an amplification system in which the alkaline phosphatase reaction provides a cofactor that activates a redox cycle leading to the formation of a colored product. In this amplification system, alkaline phosphatase dephosphorylates the reduced form of nicotinamide adenine dinucleotide phosphate, NADPH (Substrate), to reduced nicotinamide adeninedinucleotide, NADH. The NADH subsequently serves as a specific cofactor that activates a redoxcycle driven by the secondary enzyme system consisting of alcohol dehydrogenase and diaphorase (Amplifier). In the reaction catalyzed by diaphorase, NADH reduces a tetrazolium salt (INT-violet oriodonitrotetrazolium violet) to produce an intensely colored formazan dye and NAD+. NAD+in turn is reduced by ethanol, in an alcohol dehydrogenase-catalyzed reaction, to regenerate NADH, which can then re-enter the redox cycle. The rate of reduction of the tetrazolium salt and thus the amount of colored product formed are directly proportional to the amount of FGF basic bound in the initial step.

The MDD for the QUANTIKINE HS® Human FGF basic Immunoassay FGF basic ranges from 0.05 to 0.56 pg/mL. The mean MDD is 0.22 pg/mL. The minimum detectable dose is determined by adding two standard deviations to the mean optical density value of twenty zero standard replicates and calculating the corresponding concentration.

Alternative assays for the quantitation of FGF basic are based on its stimulation of the proliferation of an appropriate indicator cell line, e.g., NR6-3T3. This type of assay requires 1-2 days to complete and is not completely specific for FGF basic.

Preferably, a composition comprises two or more growth factors that synergistically interact to promote remodeling of the composition after implantation. Any combination of two or more synergistic growth factors may be used. For example, one or more growth factors can be added to an ECM material to form a composition comprising two or more synergistic growth factors. Preferably, in some embodiments, FGF-2 and VEGF growth factors are combined in a composition to synergistically promote remodeling of the implanted composition. A combination of FGF-2 and VEGF is believed to be far more potent than FGF-2 alone in inducing angiogenesis in vitro and in vivo. Furthermore, FGF-2 induces VEGF expression in smooth muscle and endothelial cells. The synergistic relationship between FGF-2 and VEGF is documented in the literature, for example in the following references which are incorporated herein in their entirety: Bootle-Wilbraham C A, et al., “Fibrin fragment E stimulates the proliferation, migration and differentiation of human microvascular endothelial cells in vitro,” Angiogenesis. 2001 ;4(4):269-75; Nico B, et al., “In vivo absence of synergism between fibroblast growth factor-2 and vascular endothelial growth factor,” J Hematother Stem Cell Res. December 2001;10(6):905-12; and Hata Y, et al., “Basic fibroblast growth factor induces expression of VEGF receptor KDR through a protein kinase C and p44/p42 mitogen-activated protein kinase-dependent pathway,” Diabetes. May 1999;48(5):1145-55.

Vascular endothelial growth factor (VEGF) is a potent and specific mitogen for vascular endothelial cells that is capable of stimulating angiogenesis during embryonic development and tumor formation. The VEGF family of structurally related growth factors has five mammalian members, VEGF, VEGF-B, VEGF-C, VEGF-D, and placenta growth factor (PIGF), all encoded by separate genes. Stacker, S. A. and Achen, M. G. “The vascular endothelial growth factor (VEGF) family: signaling for vascular development.” Growth Factors 17:1-11 (1999).

An ECM material can be tested for growth factors using any suitable assay identified by one in the art to provide the desired level of sensitivity. In some embodiments, growth factors can be identified using an in vitro assay.

Various assays for growth factors are known in the art to identify the presence of growth factors and quantify the concentration of a growth factor. For example, Human FGF Basic ELISA assay can be used to identify certain growth factors.

Other examples of growth factor assays are disclosed in U.S. Pat. No. 6,375,989 to Badylak et al., incorporated herein by reference, which discloses in vitro assays using antibodies to identify FGF-2 and TGF-beta in submucosal ECM material.

Briefly, submucosal tissue can be extracted with four different aqueous solvents and the extracts can be evaluated for their effects on Swiss 3T3 fibroblasts. Two in vitro assays may be used in parallel for the detection of factors capable of stimulating either whole cell proliferation or DNA synthesis. Specific antibodies directed against FGF-2 and TGF-beta can be used to confirm the identity of these growth factors as major fibroblast stimulating factors extractable from submucosal tissue.

It is believed that a bulking agent comprising an ECM material with one or more growth factors, such as certain submucosal tissues, induces site-specific tissue remodeling at the site of injection. To determine the components of submucosa tissue that induce tissue remodeling, submucosal tissue can be extracted and the extracts tested for the ability to stimulate Swiss 3T3 fibroblasts to synthesize DNA and proliferate. If so, each of the different extracts of submucosal tissue can have measurable growth stimulating activity when analyzed in both a whole cell proliferation assay (alamarBlue dye reduction) and a DNA synthesis assay ([³H]-thymidine incorporation). Proteins extracted from submucosal tissue with 2 M urea can induce activity profiles in the two assays which were very similar to the activity profiles of basic fibroblast growth factor (FGF-2) in the assays. As well, the changes in cell morphology in response to the extracted proteins can mimick the changes induced by FGF-2. Neutralization experiments with specific antibodies to this growth factor confirmed the presence of FGF-2 and can indicate that it was responsible for about 60% of the fibroblast stimulating activity of the urea extract of submucosal tissue.

Western blot analysis with a monoclonal antibody specific for FGF-2 can detect a reactive doublet at approximately 19 kDa and further confirm the presence of FGF-2. The activity of proteins extracted from submucosal tissue with 4 M guanidine can be partially neutralized by a TGF-beta specific antibody. Changes in the morphology of the fibroblasts exposed to this extract can be similar to changes induced by TGF-beta. Although no reactive protein band can be detected at 25 kDa in a nonreduced western blot analysis, several bands can be reactive at higher molecular weight. Identification of FGF-2 and TGFbeta-related activities in submucosal tissue (FGF-2 and TGFbeta are believed to significantly affect critical processes of tissue development and differentiation) provides the opportunity to prepare compositions for enhancing wound healing and tissue remodeling.

Other growth factors that may also be present in the ECM material include glycosaminoglycans (GAGs), chrondroitin sulfate B and Fibronectin (Fn). Assays for these growth factors are also described in U.S. Pat. No. 6,375,989, incorporated by reference above.

Glycosaminoglycans (GAGs) are important components of extracellular matrices, including submucosal tissue, and therefore extractions were performed to identify the species of glycosaminoglycans present in submucosal tissue. Without being bound by theory, GAGs are believed to represent the post-translational glycosylation of proteoglycan core proteins. Glycosaminoglycans may serve both structural and functional roles in extracellular matrices. In addition to providing structural integrity to the extracellular matrix, GAGs may modulate the healing of soft tissues in several different ways. Such modulation is believed to include organizing the deposition of collagen fibers, stimulating angiogenesis, inhibiting coagulation, and initiating cell and tissue proliferation and differentiation.

Without being bound by theory, Chondroitin sulfate B is believed to interact with growth factors as a part of an antithrombotic agent (but is also thought to have independent activity as an antithrombotic agent) by inhibiting the thrombin induced aggregation of platelets and may activate the fibrinolytic pathway by causing the release of tissue plasminogen activator (tPA). Chondroitin sulfate B may act as an anticoagulant by inhibiting thrombin formation, either directly through heparin cofactor II or antithrombin II or indirectly through protein C activation.

Fibronectin (Fn) is a large dimeric protein of the plasma and extracellular matrix with a molecular weight of approximately 440 kDa. Fn is believed to be among the first proteins deposited in new extracellular matrix and has chemotactic and cell adhesive activities for a variety of cells, including fibroblasts and endothelial cells. As these cells are important in wound healing and tissue remodeling, Fn may play a pivotal role in the recruitment and retention of host cells to the wound site. Fn comprises approximately 0.1% of the dry weight and is distributed throughout the thickness of submucosal tissue. Another growth factor is fibrin fragment E (FnE), which is believed to stimulate the proliferation, migration and differentiation of human dermal microvascular endothelial cells (HuDMECs).

Growth factors that are proteins can also be delivered to a recipient subject by incorporation in the ECM material and/or by administering to the subject as part of a composition: (a) expression vectors (e.g., plasmids or viral vectors) containing nucleic acid sequences encoding any one or more of the above factors that are proteins; or (b) cells that have been transfected or transduced (stably or transiently) with such expression vectors. Such transfected or transduced cells will preferably be derived from, or histocompatible with, the recipient. However, it is possible that only short exposure to the factor is required and thus histoincompatible cells can also be used. The cells can be incorporated into the a cellular matrices (particulate or non-particulate) prior to the matrices being placed in the subject. Alternatively, they can be injected into an a cellular matrix already in place in a subject, into a region close to an a cellular matrix already in place in a subject, or systemically. Concentrations of the various growth factors desirable in a composition will vary greatly according to the species, age, weight, size, and sex of the subject and are readily determinable by a skilled artisan.

In some embodiments, compositions disclosed herein can comprise combinations of two or more growth factors believed to act synergistically to promote remodeling of the composition upon implantation. A number of such growth factor combinations have been discovered and any grouping of growth factors thought to be synergistic to promote remodeling of the composition can be selected by one of skill in the art. Some non-limiting examples of groups of growth factors that may enhance remodeling of a composition include: FGF-2 and VEGF (see, e.g., Nico B, et al., “In vivo absence of synergism between fibroblast growth factor-2 and vascular endothelial growth factor,” J Hematother Stem Cell Res. December 2001;10(6):905-12, incorporated herein by reference); Laminin-1 and certain FGFs (See Dixelius J, et al., “Laminin-1 promotes angiogenesis in synergy with fibroblast growth factor by distinct regulation of the gene and protein expression profile in endothelial cells, “J Biol Chem. May 28, 2004;279(22):23766-72. Epub Mar. 25, 2004, incorporated herein by reference); Fibrin fragment E (FnE) and VEGF or bFGF (See Bootle-Wilbraham C A, et al., “Fibrin fragment E stimulates the proliferation, migration and differentiation of human microvascular endothelial cells in vitro,” 1: Angiogenesis. 2001;4(4):269-75, incorporated herein by reference).

Additional Components and Processing of Compositions

The compositions useful in injectable bulking agents can also include other bioactive ingredients, such as an active ingredient selected from the group consisting of osteoinductive materials, cartilage inducing factors, angiogenic factors, hormones, antibiotics, and antiviral compounds.

In some embodiments, the compositions can optionally be contacted with crosslinking agents such as glutaraledhyde, for instance to reduce the bioabsorption.

The compositions can be sterilized using art-recognized sterilization techniques. Materials of the present invention are typically derived by admixing collagen, water and an acid. As discussed above, the material can also include other substances such as an active ingredient. Chemical treatment using peracetic acid (PAA) and dialysis as known in the art can be used to sterilize the compositions disclosed herein. The material can also be sterilized by dialysis, irradiation (e.g. using g-radiation), filtration, chemical treatment (e.g., using ethylene oxide), or other known sterilization methods. Alternatively, the material which can be a gel is lyophilized to a dry solid before being sterilized. When sterilizing the material using a chemical treatment, it is preferred that the material be lyophilized to a dry solid prior to being sterilized. Lyophilization removes water and prevents any chemical reaction which may occur between the chemical used for sterilization (e.g., ethylene oxide) and water. Another alternative method is to make the material of the present invention in an aseptic environment, thereby eliminating the need for a separate sterilization step.

The compositions can be provided in any suitable form, including without limitation a powder or a particulate suspension. In some embodiments, the compositions can be prepared as a suspension. The size of the particles chosen for a particular application will be determined by a number of factors. Smaller particles are easier to inject with a smaller gauge size needle; however, embolization due to migration of the particles is a concern with the smaller particle sizes. The size of the particles used in a particular procedure will include consideration of the procedure employed, disease progression, the degree of degradation of the affected region, patient size, the disposition of the patient, and the preferences and techniques of the doctor performing the procedure. Similarly, such factors must be considered when determining the proper volume of bulking agent to inject into a patient. Preferably, the solids are microspheres having diameters large enough to minimize immediate phagocytosis by macrophages and intra-capillary diffusion, or greater than about 10 μm. On the other hand, solid particulate diameters small enough to achieve a desired texture of the injectable and facilitate the free flow of the injectable through intradermal needles (typically 26-28 gauge) is also preferred.

In a particular embodiment, the bulking agent comprises a suspension of solid particles comprising a composition as described above, and sized in a range of about 10 microns to about 1500 microns in diameter, preferably about 150 microns to about 1100 microns in diameter, and more preferably about 500 microns to about 900 microns in diameter.

For example, in one embodiment, the particulate suspension can have a concentration of solids of from about 1 mg/mL to about 200 mg/mL. The particulate suspension can have any suitable concentration of solids, including 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 110, 120, 130, 140, 150, 160, 170, 180, 190, 200 mg/mL or higher. Preferably, the particulate suspension has a concentration of solids of between about 1 mg/mL and about 100 mg/mL, and more preferably between about 4 mg/mL and 50 mg/mL.

One preferred particulate suspension composition may comprise a concentration of at least 2.0 ng/mL of a growth factor. In one embodiment, the particulate suspension composition comprises solid particles with diameters on the order of about 10 and 1,000 microns (0.01-1.0 mm). A suspension of solid particles of the compositions described above can be administered by periurethral or transurethral injection can increase pressure on the urethra and compress the urethral lumen, thus alleviating urinary incontinence by enhancing urethral resistance to the flow of urine. Similarly, a suspension of such cells that is administered by injection into tissues adjacent to the ureteral orifice can increase support behind a refluxing intravesical ureter, thus alleviating vesicoureteral reflux by providing resistance to urinary reflux.

Injectable Bulking Agents

In some embodiments, the compositions are prepared as injectable bulking agent formulations. The bulking agents preferably comprise the compositions described above. Preferably, the injectable bulking agent formulations comprise particles of ECM material containing telocollagen and a growth factor. The compositions can include any detectable amount of telocollagen and one or more growth factor. In some aspects, the compositions can comprise both telocollagen and a telocollagen. Preferably, the composition can be formulated into an injectable bulking agent solution or suspension comprising at least 10 ng/mL of telocollagen and at least 2.0 ng/mL of at least one growth factor. In some embodiments, an injectable bulking agent can include between about 10 ng/mL and about 200 mg/mL, such as 0.1 μg/mL, 1.0 μg/mL, 10 μg/mL, 100 μg/mL, 1 mg/mL and 10 mg/mL of telocollagen and any incremental amount therebetween. Preferably, the injectable bulking agent comprises between about 1 mg/mL and 200 mg/mL of collagen, where at least a portion of the collagen includes telocollagen and optionally includes a telocollagen.

The compositions can further comprise a variety of other compounds, including without limitation: carriers, gelling agents, polymers, cryoprotecting agents, surfactants, tensoactive agents, and buffering agents. The compositions can be combined with any suitable liquid, gel or solid carrier or vehicle. Examples of suitable liquid carriers include phosphate-buffered physiological saline, PBS, water, saline, Krebs-Ringer solution containing 5% dextrose, or in any other physiological solution. Examples of suitable gel carriers include gelatin powder, such as denatured porcine collagen types I and III, and water-based gelling agents.

In some embodiments, the injectable bulking agent may also comprise gelling agents. Gelling agents are well known in the art and are ingredients that aid gel formation. Suitable gelling agents include, but are not limited to, cellulose derivatives, such as hydroxypropylmethylcellulose (“HPMC”) and carboxymethylcellulose (“CMC”), synthetic hyaluronic acids, lactic acid esters, sodium carmellose, caproic acid esters, and the like.

The concentration of the gelling agent in the activated form will vary depending upon the intended application, but, may typically vary from about 0-10% by weight, more typically from about 1% to about 5% by weight, with from about 2% to about 3% by weight being preferred. The pre-activated powder form for the injectable may typically comprise from about 0-40%, preferably from about 20% to about 30%, or from about 22% to about 26%, by weight gelling agent, if any. The amount of gelling agent is typically chosen to obtain a suspension having the desired flow properties, i.e., not too thick or gelatinous or too liquid.

For some embodiments, the injectable bulking agent may also contain a cryoprotecting agent. A cryoprotecting agent is a chemical which inhibits or reduces the formation of damaging ice crystals in biological tissues during cooling. Suitable cryoprotecting agents include, but are not limited to sugars and carbohydrates, such as d-mannitol, lactose, sucrose, fructose, and dextran, with d-mannitol being preferred. The concentration of the cryoprotecting agent in the activated suspension to be injected will vary depending upon the intended application, and particulars related to the bulking agent composition and solid particles therein, identity of the cryoprotecting agent, but will vary from about 0-50 mg per 100 mL of suspension, typically from about 27 to about 35 mg per 100 mL of the pharmaceutically acceptable carrier, with concentrations in the range of from about 29 to about 32 mg per 100 mL of the pharmaceutically acceptable carrier being preferred. The lyophilized powder form for the injectable may typically comprise 0-45% by weight, or from about 30% to about 40% or from about 33% to about 38% or about 35% by weight, cryoprotecting agent, if any.

For some embodiments, the injectable bulking agent may also contain a surfactant or tensoactive agent. A surfactant is a chemical that reduces the surface tension in a solution, allowing small, stable bubbles to form. Suitable surfactants include, but are not limited to, polysorbates, such as polyoxyethylene sorbitans, or pluronic acid, preferably polyoxyethylene sorbitan monooleate, polyoxyethylene sorbitan trioleate, polyoxyethylene sorbitan monostearate, polyoxyethylene sorbitan tristearate, polyoxyethylene sorbitan monopalmitate, or polyoxyethylene sorbitan monolaurate, with polyoxyethylene sorbitan monooleate (Tween 80™), polyoxyethylene sorbitan monostearate (Tween 60™), and polyoxyethylene sorbitan monolaurate (Tween 20™) being preferred, and polyoxyethylene sorbitan monooleate (Tween 80™), being even more preferred.

In these embodiments, the surfactant is typically present in the activated form of the implant in a concentration of from 0-0.03% by weight, more typically from about 0.019% to about 0.024%, preferably about 0.021%. The lyophilized powder form for the injectable may comprise from 0-0.3%, preferably from about 0.22% to about 0.27% or about 0.24% by weight surfactant, if any.

For some embodiments, the injectables may also contain a buffering agent. A buffering agent is a chemical compound or, compounds that is added to the solution to allow that solution to resist changes in pH as a result of either dilution or small additions of acids or bases. Effective buffer systems employ solutions which contain large and approximately equal concentrations of a conjugate acid-base pair (or buffering agents). The buffering agents employed herein may be any such chemical compound(s) which is pharmaceutically acceptable, including but not limited to salts (conjugates acids and/or bases) of phosphates and citrates. The concentration of the buffering agent(s) will depend upon its strength, the composition of the implant and its intended purpose, but may typically range in the activated form from about 0-0.1 mg per 100 mL of the pharmaceutically acceptable carrier, or from about 0.08 mg to about 0.1 mg per 100 mL of the pharmaceutically acceptable carrier, with about 0.09 mg per 100 mL of suspension being preferred. The lyophilized powder form for the injectable may typically comprise from 0-0.2% by weight, or from about 0.09% to about 0.11% by weight buffering agent, if any.

An injectable bulking agent may also optionally contain one or more additional medicaments. As used herein, a “medicament” may be any bioactive composition for therapeutic administration with the ECM material compositions disclosed herein. In some embodiments, an additional medicament is provided as part of an ECM material composition. In other embodiments, an additional medicament is administered separately from the administration of the ECM material.

A medicament composition includes, but is not limited to, any pharmaceutical compound which one desires to administer to a subject receiving an injection of the implant. Preferably, the medicament is administered by injection as part of the injectable implant composition. In some embodiments, the bioactive is selected from the group consisting of: physiologically compatible minerals, antibiotics, chemotherapeutic agents, antigen, antibodies, enzymes, anesthetics, thrombolytics, vasodilators, antihypertensive agents, antimicrobials or antibiotics, antimitotics, antiproliferatives, antisecretory agents, non-steroidal anti-inflammatory drugs, immunosuppressive agents, hormones, growth factor antagonists, antitumor and/or chemotherapeutic agents, antipolymerases, antiviral agents, photodynamic therapy agents, antibody targeted therapy agents, prodrugs, free radical scavengers, antioxidants, biologic agents, radiotherapeutic agents, radiopaque agents and radiolabelled agents. Preferably, in some embodiments, the medicament optionally comprises an anesthetic to decrease the pain or discomfort associated with injecting the implant or a composition that facilitates the integration of the polymer or decreases the trauma to the injection site. Exemplary anesthetics include but are not limited to lidocaine, xylocaine, novocaine, benzocaine, prilocaine, ripivacaine, and propofol. The medicament may be included in an injectable bulking agent in any suitable manner, including adding the medicament to the injectable bulking agent during manufacturing or prior to the injection during activation mixing with a pharmaceutically acceptable carrier. Preferably, the injectable bulking agent can comprise a therapeutically effective amount of a medicament. For instance, an injectable bulking agent can comprise about 0.1 % to about 0.5% of an anesthetic such as lidocaine, or more preferably about 0.3% lidocaine.

In some embodiments, the bulking agents disclosed herein may further comprise a biodedgadable polymer such as glycolic acid (GA) and polymers containing lactic acid repeat units (also referred to herein as PLA). One skilled in the art can select suitable biodegradable polymers where appropriate. Some factors affecting the selection of suitable biodegradable polymers include monomer selection, initiator selection, process conditions, and the presence of additives. These factors in turn influence the polymer's hydrophobicity, crystallinity, melt and glass-transition temperatures, molecular weight, molecular-weight distribution, end groups, sequence distribution (random versus blocky), and presence of residual monomer or additives. In some embodiments, the bulking agents comprise glycolic acid (“GA”) monomer and biocompatible, biodegradable particles of polymers comprising lactic acid (“PLA”). The injectable bulking agent may comprise, for example, PLA particles, preferably microspheres, having a diameter ranging primarily from about 20 μ, to about 120 μ, typically from about 40 μm to about 80 μ, preferably with a mean diameter of approximately 60 μ. For some embodiments, it is preferred to employ microspheres having diameters larger than about 40 μ, to minimize immediate phagocytosis by macrophages and intra-capillary diffusion. Preferably, solid particle diameters are selected to produce a desired texture of the injectable bulking agent and facilitate the free flow of the injectable through intradermal needles (typically 26-28 gauge).

In some embodiments, the bulking agents disclosed herein may further comprise a non-biodedgadable polymer such as methacrylate polymer including methacrylate and methylmethacrylate. For example, the bulking agent may comprise a composition described above and about 10% Methacrylate. Any suitable biocompatible polymer or polymer amount may be used.

In one embodiment, bulking particles are injected through a needle. In other embodiments, a cystoscope is used to allow for viewing the injection area. The bulking particles can be supplemented with a contrast agent to enhance their appearance as an aid to the doctor performing the procedure. Other methods of visual enhancement to assist in viewing of the bulking agent can also be employed. Injection of the particles can also be accomplished transuretherally by, for example, using a catheter.

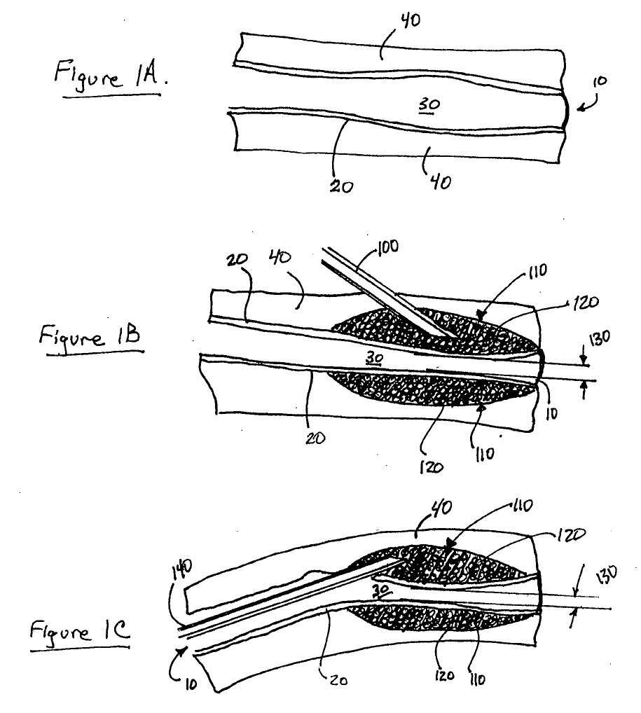

FIG. 1A depicts a side view of a tissue structure with an enlarged lumen surrounded by muscle tissue. A body passage 10, having a wall 20 and an enlarged lumen 30 surrounded by muscle tissue 40 is shown in side view. FIG. 1B depicts the tissue structure of FIG. 1A immediately after injection of a bulking agent around the enlarged lumen of the tissue. The body passage 10 is shown immediately after a bulking agent has been injected around the enlarged lumen 30 of the tissue. A hypodermic needle 100 is inserted through the tissue 40, preferably near the enlarged lumen 30, stopping near the wall 20 of the enlarged lumen 30. Thereafter, a bulking agent 110 including solid particles 120 is injected via the hypodermic needle 100 into the tissue 40 adjacent the wall 20. The result is a constricted region 130 located in the vicinity of the accumulation of the bulking agent 110. Alternatively, referring to FIG. 1C, the body passage 10 is shown immediately after the bulking agent 110 has been injected around the enlarged lumen 30 of the tissue 40. An elongate needle 140 may be inserted from within the body passage 10 and into surrounding tissue 40.

Various needles, preferably from a 16 to 28 gauge, can be used to dispense the bulking composition without clogging. In some applications, a smaller range of needle sizes may be preferred, for example 18-22 gauge.

In a particular embodiment, the bulking agent comprises a suspension of solid particles comprising a composition as described above. The size of the particles chosen for a particular application will be determined by a number of factors. The size of the particles used in a particular procedure will include consideration of the procedure employed, disease progression, the affected region, patient size, the disposition of the patient, and the preferences and techniques of the doctor performing the procedure. Similarly, such factors must be considered when determining the proper volume of bulking agent to inject into a patient. In one embodiment of the invention, the volume of bulking composition is about 1 mL to about 30 mL, and preferably about 20 mL to about 30 mL. In another embodiment, the volume of bulking composition injected into a patient is about 2 mL to about 16 mL. However, these amounts can vary significantly based on the doctor's determination as to when the target region is sufficiently bulked up.

In some embodiments, lyophilized particles of ECM material are provided. The total surface area (sum of internal and external surface area) of the particles of ECM in an injectable bulking agent can be measured, for example, by BET surface area analysis. In some embodiments, the total surface area of the ECM material particles preferably is greater than 0.1 m²/g, more preferably greater than or equal to 0.2, 0.3, 0.4, 0.5, 0.6, 0.7, 1.0, 1.1, 1.2, 1.3, 1.4, 1.5, 1.6, 1.7, 1.8, 1.9, or 2.0 m²/g. This level of total surface area provides sufficient surface area to enhance wetting of the dry porous matrix and enhance drug dissolution. Preferably, the ECM material particles comprise one or more growth factors. More preferably, the ECM material particles comprise telocollagen. Most preferably, the lyophilized particles can be combined with a liquid vehicle to form a suspension of particles useful as an injectable bulking agent. In one embodiment, lyophilized ECM particles comprising one or more growth factors and telocollagen are combined with a liquid vehicle to form a suspension with a concentration of solid ECM particles of about 1, 5, 10,15, 20, 25, 30, 35, 40, 45, 50, 55, 60, 65, 70, 75, 80, 85, 90, 100, 110, 120, 130, 140, 150, 160, 170, 180, 190 or 200 mg/mL of the suspension.

In one embodiment, the bulking agent comprises a composition disclosed above dispersed in a phosphate buffered physiological saline with a pH of about 7.3 and a solid concentration of about 35 mg/mL.

In another embodiment, the bulking agent comprises a composition disclosed above and further comprises a non-biodegradable polymer such as zirconium oxide or polymethylmethacrylate (PMMA) beads with diameters of about 200 to about 500 microns suspended in a water based carrier gel. The non-biodegradable polymer beads can optionally be coated with pyrolytic carbon. The bulking agent can also comprise beta-glucan.

In yet another embodiment, the bulking agent further comprises sodium hyaluronate.

In another embodiment, a bulking agent may optionally further comprise 0.3% lidocane or 3 mg/mL of lignocaine.

In one embodiment, the bulking agent comprises about 40% or more hyaluronic acid, and optionally further comprises about 10% selenium CS, 10% vanadium CS, 10% zinc CS.

In one embodiment, the bulking agent can further comprise calcium hydroxyapatite (CaHA), for example as microspheres suspended in a polysaccharide gel.

Kits

Materials of the present invention can be part of a kit containing the components of the materials. Such kits are particularly useful for health care professionals in preparing the materials and compositions of the present invention immediately before use. Such kits, in addition to including the component parts of the various materials and compositions of the invention can also include one or more containers for mixing the components, along with optional mixing devices such as stirrers. Further, such kits can include the components in sealed, pre-measured packages. The sealed packages can be sealed aseptically and the amounts of the components can be pre-measured in relative amounts as described elsewhere herein.

The injectable bulking agent of the present invention may be typically provided in a ready for use prefilled sterile syringe, or in a vial in the form of a sterile suspension. In preferred embodiments, the injectable bulking agent may also comprise a composition described above packaged in vials as a freeze-dried, free-flowing powder. Once activated with distilled injectable water or other pharmaceutically acceptable carrier prior to injection, the gelatinous (suspension) fluid may be implanted by subcutaneous injection. In some embodiments, the end user may also add additional components besides the pharmaceutically acceptable carrier to the bulking agent prior to injection. The injectable may also be provided in a two-compartment prefilled syringe, one containing the freeze-dried powder and the other containing water or other pharmaceutically acceptable carrier. If reconstituted extemporaneously, e.g., by double distilled water, for injectable preparations, the gel-like fluid (suspension) may then be applied by intradermal or subcutaneous injection. The viscosity of the suspension is inversely proportional to temperature.

In some embodiments, kits provide devices and systems for injecting bulking agents. In one embodiment, the present invention also relates to kits comprising devices used to dilate tissue within a treatment tissue region to facilitate injection of the bulking agent. For example, the kits can include: a needle with a penetration device (e.g., a taper point obtuator or trocar); a penetration device; a balloon portion for advancement through the needle and subsequent inflation of the balloon within the tissue to create a void, and a syringe with a bulking agent to be joined to the needle and injecting the bulking agent into the tissue void. This procedure can be repeated as necessary in order to maximize the effectiveness of the bulking agent and to achieve the desired results.

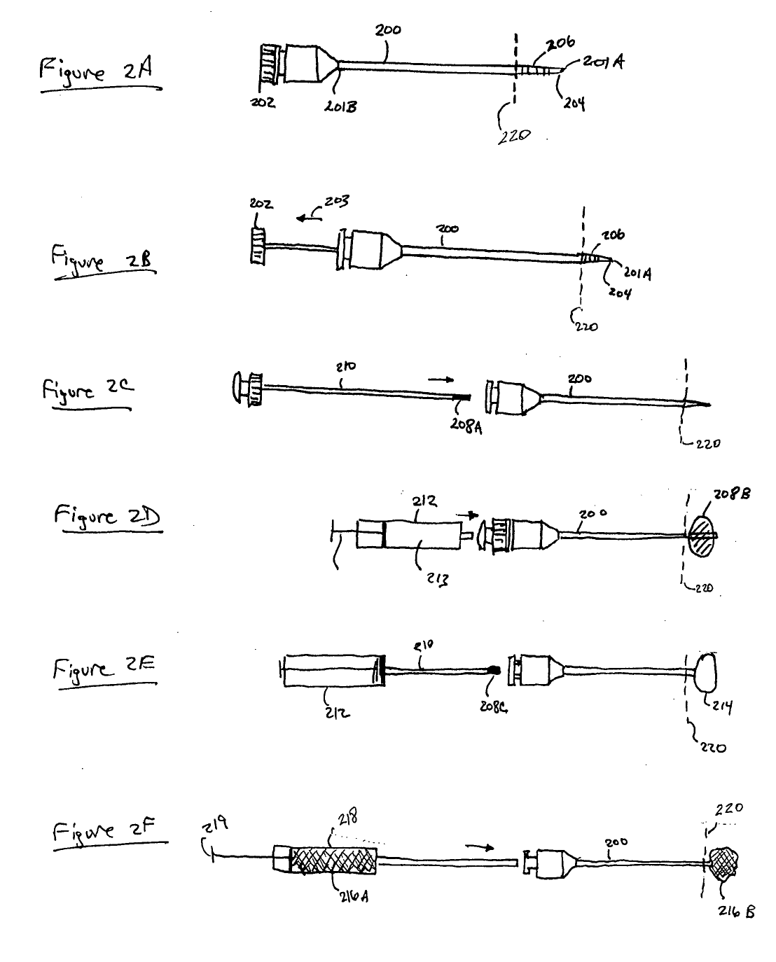

In one embodiment, the kit of FIG. 2A can be used to inject a bulking agent, for example, as described below. A needle 200, such as a blunt-end hypotube or hypodermic needle having a distal end 201A and a proximal end 201B, is adapted to accept a penetration device 204, such as a taper point obtuator or a trocar, at the distal end 201A of the needle 200. The needle 200 may range in size from about 18 gauge to about 22 gauge, and preferably about 20 gauge to about 22 gauge. The penetration device 204 is attached to the needle 200 to enable penetration of the needle 200 into the tissue. The penetration device 204 may be adapted to the needle 200 by way of a luer hub or fitting 202, and in one embodiment, a male luer hub is used. The needle 200 is inserted with the penetration device 204 into the treatment region (e.g., a sphincter region) to the desired depth 220 within the tissue of a subject being treated. In one embodiment, desired penetration depth can be determined by indicia such as striping 206 located on the penetration device 204. In one embodiment, the amount of penetration of the penetration device 204 ranges from about 2 cm to about 2.5 cm. In one embodiment, the amount of tissue penetration of the needle 200 ranges from about 0.5 cm to about 1 cm beyond the tissue line 220. Next, the penetration device 204 is removed while retaining the inserted needle 200 (FIG. 2B).

A luer hub 202 or fitting, or in one embodiment a female luer hub, may be adapted to the proximal end 201B of the needle 200, to which a syringe 212, 218 (FIGS. 2D-2F) is adapted. Referring to FIG. 2A, the luer hub 202 is depicted in its locked position, while in FIG. 2B the luer hub 202 is depicted in its unlocked and retracted 203 position. In the locked position, the luer hub 202 can be positioned for inflating a balloon 208A or injecting a bulking agent 216A, 216B. As shown in FIG. 2C, the luer hub 202 in the unlocked and retracted position can be positioned for accepting the balloon 208A for insertion or for removal of the balloon 208C after dilation 208B.

The balloon 208A (pre-inflation) is adapted to advance through a lumen of the needle 200, and an adapter on the balloon provides a means to lock the pre-inflated balloon 208A to the luer hub 202, which in turn connects to the syringe 212 (FIG. 2D). The pre-inflated balloon 208A is then inflated 208B (inflated) using an inflation device, such as the syringe 212 injecting an inflation medium 213 to inflate the balloon 208B. Inflation of the balloon 208B creates a void 214 in the treatment region (FIG. 2E). The balloon is then deflated 208C (deflated, post-inflation) and removed from the treatment region, resulting in a tissue void 214 that the inflated balloon 208B previously occupied. As shown in FIG. 2E, the deflated balloon 208C is removable through the lumen of the needle 200. In one embodiment, a plastic tube or other tip 210 is used to aid in removal of the deflated balloon 208C.

A syringe or other injection device 218 containing the bulking agent 216A is then affixed to the needle 200 (FIG. 2F). The plunger of the syringe 219 is then depressed, thereby injecting the bulking agent 216A into the tissue void 214, creating an injected mass of bulking agent 216B filling the void 214.

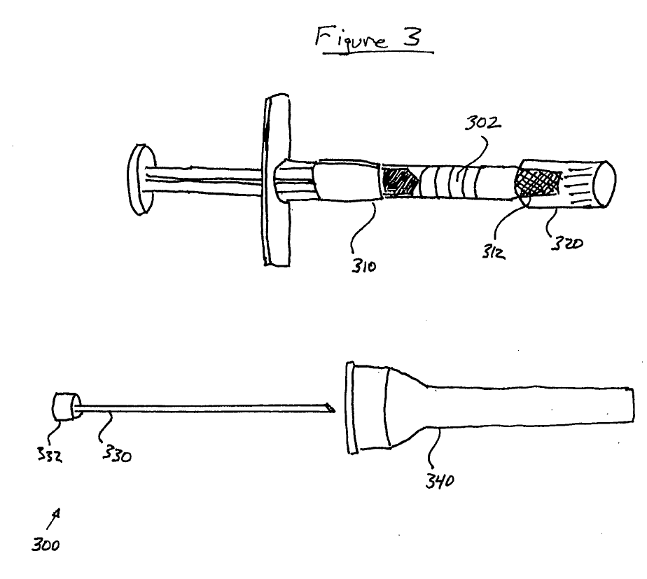

Another embodiment provides a single-use injectable bulking agent injection kit 300, as shown in FIG. 3. An injectable bulking agent 302 comprising a suspension of SIS particles with a growth factor and telocollagen is prepackaged in a syringe 310. The distal portion 312 of the syringe 310 is sealed with a protective cap 320. Also provided are a delivery needle 330 enclosed in a removable safety cap 340 adapted to enclose the delivery needle 330. A fitting at the proximal end 332 of the delivery needle 330 is adapted for coupling to the distal portion 312 of the delivery syringe 310 after removal of the protective cap 320. In one embodiment, the injectable bulking agent injection kit 300 can be used according to the following steps: the protective cap 320 is removed from the syringe 310, the distal portion 312 of the delivery syringe 310 is coupled to the fitting at the proximal end 332 of the delivery needle 330, the delivery needle 330 is removed from the removable safety cap 340, the injectable bulking agent 302 is injected into a patient, and properly disposed of.

In one embodiment, a bulking agent is provided in individually packaged syringes each containing about 2.5 mL of bulking agent intended for single use. The contents of the syringes are sterile and nonpyrogenic.

In another embodiment, the kit can comprise pre-packaged syringes with about 2.5 cc of a bulking agent in each syringe and two 30 gauge needles.

In some embodiments, the kit comprises a first vessel containing 100 mg of freeze-dried bulking agent comprising porcine derived Si, a second vessel containing about 125 mg of e-aminocaproic acid. The kit optionally comprises directions for the mixing the contents of the first vessel and the second vessel with a suitable liquid vehicle to prepare an injectable bulking agent formulation.

In some embodiments, the kit comprises one or more syringes containing about 2.5 mL of bulking agent with about 60 mg of collagen per mL.

Methods of Treatment

Another aspect of the present invention includes methods of treating various conditions comprising implantation composition as broadly described above into a body. While most uses of these compositions are concerned with human application, the methods of treatment are applicable to a wide variety of animals, particularly mammals. As used in this invention, the term “implanting” refers to placing a composition such as an injectable bulking agent in an area in which it is desired, for example to provide a tissue mass. Such methods of implantation can involve a surgery or a simple injection of the product using any of the known methods including a use of syringe.

As used herein, the term “placing” a composition includes, without limitation, setting, injecting, infusing, pouring, packing, layering, spraying, and encasing the composition. In addition, placing “on” a recipient tissue or organ means placing in a touching relationship with the recipient tissue or organ.

The suspension can be injected via a syringe and needle directly into a specific area wherever a bulking agent is desired.

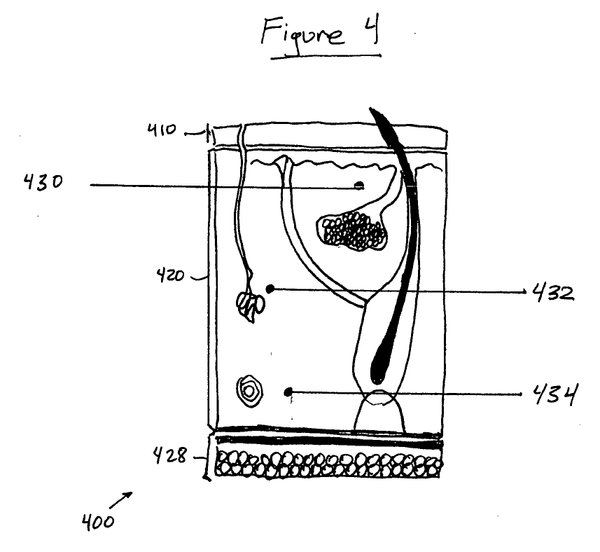

For example, a bulking agent can be used to treat a soft tissue deformity such as that seen with areas of muscle atrophy due to congenital or acquired diseases or secondary to trauma, burns, and the like. FIG. 4 illustrates typical injection sites in the dermis for cosmetic and lipodystrophy methods. A cross section of human facial tissue 400 shows the epidermis 410 attached to connective tissue 420 with an underlying subcutaneous adipose layer 428. Typical injection sites for a bulking agent include a first site for treating acne scars or small facial wrinkles 430, a second site for treating more pronounced wrinkles, creases and reshaping of a facial profile, and a third site 434 for treating lipodystrophy.

The invention also provides methods for administering compositions of the invention for augmentation and/or repair of dermal, subcutaneous, and fascial tissues. Compositions containing the compositions and bulking agents described above, with or without passaged muscle cells, matrix components, and/or fillers can be injected or implanted into a subject to treat, for example, scarring, cellulite, skin laxness or skin thinning, wrinkles, wounds (e.g., acute, chronic, partial or full-thickness wounds, burns, pressure sores, and ulcers), breast deficiencies, periodontal disorders, defects of an oral mucosa, trauma to an oral mucosa, diabetes, venous stasis, hernias, damage to ligaments, tendons and muscles of the joints, and allopecia. Methods for treating these conditions can involve, for example, injecting into the site of the deficiency or defect a composition that contains autologous, passaged fibroblasts and passaged muscle cells (e.g., autologous, passaged muscle cells), wherein the cells are substantially free of culture medium serum-derived proteins.

The suspension can also be injected as a bulking agent for hard tissue defects, such as bone or cartilage defects, either congenital or acquired disease states, or secondary to trauma, burns, or the like. An example of this would be an injection into the area surrounding the skull where a bony deformity exists secondary to trauma. The injunction in these instances can be made directly into the needed area with the use of a needle and syringe under local or general anesthesia.

The suspension could also be injected percutaneously by direct palpation. The suspension could also be injected through a catheter or needle with fluoroscopic, sonographic, computed tomography, magnetic resonance imaging or other type of radiologic guidance. This would allow for placement or injection of this substance either by vascular access or percutaneous access to specific organs or other tissue regions in the body, wherever a bulking agent would be required.

Further, this substance could be injected through a laparoscope or thoracoscope to any intraperitoneal or extraperitoneal or thoracic organ. For example, the suspension could be injected in the region of the gastroesophageal junction for the correcting of gastroesophageal reflux. This could be performed either with a thoracoscope injecting the substance in the esophageal portion of the gastroesophageal region, or via a laparoscope by injecting the substance in the gastric portion of the gastroesophageal region, or by a combined approach.

In cases of acid reflux, the bulking agents may be used to treat a deficiency of the pyloric sphincter. Gastroesophageal reflux disease (GERD) involves the regurgitation of stomach gastric acid and other contents into the esophagus or diaphragm. Atypical manifestations of GERD include: asthma; chronic cough; laryngitis; sore throat; and non-cardiac related chest pain.

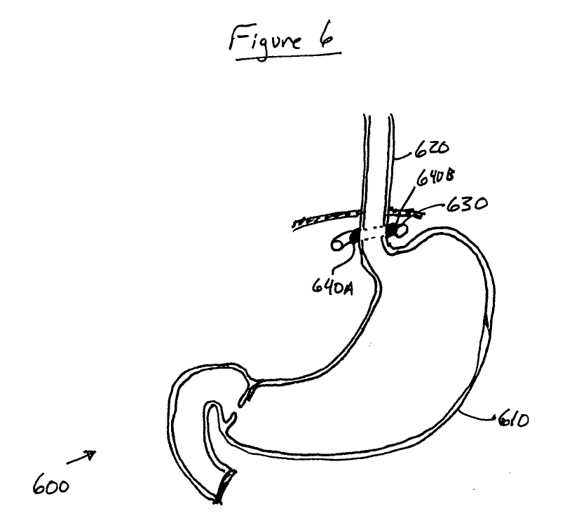

GERD can be treated by injecting a suspension of the compositions described above adjacent to the lower esophageal sphincter. FIG. 6 illustrates typical injection site for the treatment of lower esophageal sphincter deficiency. The esophagus 630 is joined to the stomach 610 at an esophageal sphincter muscle 630. In one embodiment, GERD can be treated by injection of a bulking agent at symmetric sites near the esophageal sphincter 630, such as at first injection site 640A that is positioned on the opposite side of the esophagus from a second injection site 640B.

In addition to its use for the endoscopic treatment of reflux, the system of injectable autologous muscle cell may also be applicable for the treatment of other medical conditions, such as urinary and rectal incontinence, dysphonia, plastic reconstruction. The suspension can be injected through a cystoscopic needle, having direct visual access with a cystoscope to the area of interest, such as for the treatment of vesico-ureteral reflux or urinary incontinence.

Methods of the invention can be used to treat urinary incontinence and/or vesicoureteral reflux by reforming or repairing tissue (e.g., sphincter structures) surrounding the urethra, ureters, and esophagus, thus causing a reduction in size of abnormally wide and loose lumens. These methods involve placement (e.g., injection or implantation) of compositions of the invention into the regions surrounding the urethra, ureters, or esophagus, or directly into a pocket created in the region to be repaired or augmented.

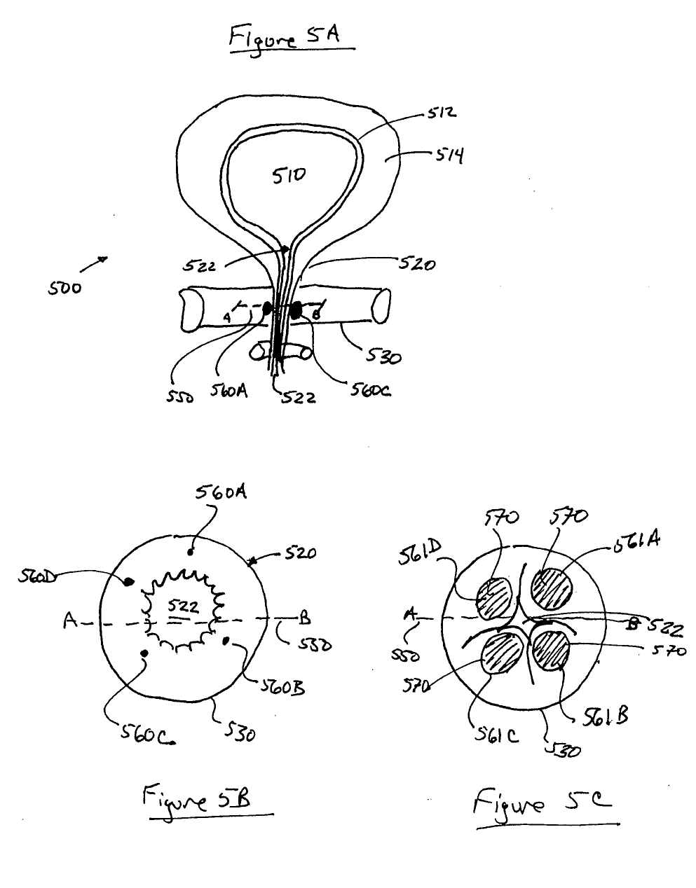

FIGS. 5A, 5B and 5C illustrate typical injection sites for the treatment of urethral sphincter deficiency. A bladder 500 includes an internal cavity 510 surrounded by a lining 512, muscle 514 and other types of tissue. The urethra 522 is formed through the neck of the bladder 520 and is surrounded by a sphincter muscle 530 that provides urinary continence. A cross section of the urethra 522 within the sphincter muscle 530 is shown at 550A-B. FIG. 5B is a cross section of the urethra 522 and surrounding tissue of the sphincter muscle 530 along cross section line 530A-B. A first injection site 560A, a second injection site 560B, a third injection site 560C and a fourth injection site 560D are symmetrically arranged around the urethra 522. A bulking agent comprising a suspension of solid particles can be administered by periurethral or transurethral injection to increase pressure on the urethra and compress the urethral lumen, thus alleviating urinary incontinence by enhancing urethral resistance to the flow of urine. Similarly, a suspension of such cells that is administered by injection into tissues adjacent to the ureteral orifice can increase support behind a refluxing intravesical ureter, thus alleviating vesicoureteral reflux by providing resistance to urinary reflux. FIG. 5C shows the cross sectional view of FIG. 5B, including the cross sectional line 550A-B, after injection of a bulking agent 570 to form a first injected mass 561A, a second injected mass 561B, a third injected mass 561C, and a fourth injected mass 561D to provide increased pressure on the urethra 522.

Erectile dysfunction (ED), or the consistent inability to maintain an erection, is generally categorized as: organic, psychogenic, or both (organic and psychogenic). Organic ED is the result of an acute or chronic physiological condition, including endochrinologic, neurologic or vascular etiologies. Thus, an aspect of the present invention encompasses using the disclosed bulking agents to treat ED. A typical procedure involves injecting the bulking agent directly at the deep fascia throughout the length of the corpus cavernosum.

In addition to the use of the cell-polymer suspension for the treatment of reflux and incontinence, the suspension can also be applied to reconstructive surgery, as well as its application anywhere in the human body where a biocompatible injectable bulking agent material is necessary. The suspension can be injected endoscopically, for example through a laryngoscope for injection into the vocal chords for the treatment of dysphonia, or through a hysteroscope for injection into the fallopian tubes as a method of rendering the patient infertile, or through a proctoscope, for injection of the substance in the perirectal sphincter area, thereby increasing the resistance in the sphincter area and rendering the patient continent of stool.

This technology can be used for other purposes. For example, custom-molded cell implants can be used to reconstruct three dimensional tissue defects, e.g., molds of human ears could be created and a chondrocyte-hydrogel replica could be fashioned and implanted to reconstruct a missing ear. Cells can also be transplanted in the form of a thee-dimensional structure which could be delivered via injection.

An aspect of the present invention encompasses the use of the injectable implants disclosed herein as a bulking agent for intracordal injections of the laryngeal voice generator by changing the shape of this soft tissue mass.

The present invention also relates to compositions and methods for providing controlled release of beneficial pharmaceutically active agents or medicaments.

### EXAMPLES

The following examples are included to demonstrate preferred embodiments of the invention. It should be appreciated by those of skill in the art that the materials and techniques disclosed in the examples which follow represent materials techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation of Injectable SIS Suspension

Cryoground SIS particulates were digested in acid (pH=2.0) for 120 hours at 4° C. in the absence of pepsin, resulting in partial digestion of the SIS particulates. The partially digested SIS particulates were centrifuged at 15000G for 45 minutes. The supernatant of soluble telocollagen was removed from the centrifuged composition. The pellet was usable as an injectable after raising the pH to physiological levels. The solid content of the partially digested particulates was approximately 35 mg/mL.

### Example 2

### alternative Preparation of Injectable SIS Suspension with Partial Digestion

Cryoground SIS particulates are partially digested in acid without pepsin (pH=2.0) for about 96 hours, then pepsin was added to further digest the SIS particulates for 24 hours, at 4° C., resulting in partial digestion of the SIS particulates. The partially digested SIS particulates were centrifuged at 15000G for 45 minutes. The supernatant of soluble telocollagen was removed from the centrifuged composition. The yield of telocollagen in the pellet was considerably less due to the excessive solubilization of collagen by pepsin.

### Example 3

### Preparation of Injectable Bulking Agent from Digested SIS

The pellet of Example 1 was lyophilized and reconstituted with PBS to produce injectable bulking agents with solid concentrations of 100 mg/mL and 150 mg/mL lyophilized solid in solution.

### Example 4

### Human FGF basic Immunoassay

The pellet of Example 1 was lyophilized and reconstituted with PBS up to solid concentrations of 50 mg/mL to produce an injectable bulking agent. The presence of at least 1 ng/mL of FGF-2 growth factor in the injectable bulking agent solution was detected using a QUANTIKINE HS® Human FGF basic Immunoassay, which is a quantitative sandwich enzyme immunoassay generically described in the description above.

I claim:

1. A composition comprising lyophilized particles of extracellular matrix material comprising telocollagen and a first growth factor, wherein an acetic acid solution comprising 50 mg of the lyophilized particles per mL of 0.5 M acetic acid is characterized by a concentration of at least 1.0 ng/mL of the first growth factor and between about 10 ng and about 200 mg of telocollagen per mL of the acetic acid solution.

2. The composition of claim 1, wherein the extracellular matrix material is selected from the group consisting of: small intestine submucosa (SIS), renal capsule matrix (RCM) and urinary bladder matrix (UBM).

3. The composition of claim 1, wherein the extracellular matrix material is porcine SIS and the acetic acid solution comprises between about 1 mg/mL and about 100 mg/mL of telocollagen.

4. The composition of claim 1, wherein the acetic acid solution is characterized by a concentration of at least 10.0 ng/mL of FGF-2 growth factor.

5. The composition of claim 1, where the first growth factor is selected from the group consisting of: a fibroblast growth factor, a vascular endothelial growth factor, a platelet derived growth factor, an insulin-like growth factor, a placenta growth factor and a transforming growth factor.

6. The composition of claim 1, where the first growth factor is selected from the group consisting of: FGF1, FGF2, FGF3, FGF4, FGF5, FGF6, FGF7, FGF8, FGF9, FGF10, VEGF A, VEGF B, VEGF C, VEGF D, and VEGF E, PIGF, PDGF, EGF, IFN-alpha, IFN-beta, or IFN-gamma, TGF-alpha, and TGF-beta.

7. The composition of claim 1, wherein the first growth factor is FGF-2.

8. The composition of claim 7, wherein the composition further comprises a vascular endothelial growth factor.

9. An injectable bulking agent comprising a suspension of particles containing an extracellular matrix material comprising telocollagen and a first growth factor, the particles suspended in a liquid vehicle to form a suspension characterized by a concentration of at least 1.0 ng/mL of the first growth factor in the suspension.

10. The injectable bulking agent of claim 9, wherein the extracellular matrix material is selected from the group consisting of: small intestine submucosa (SIS), renal capsule matrix (RCM) and urinary bladder matrix (UBM).

11. The injectable bulking agent of claim 9, wherein the suspension has a concentration of particles of from about 1 mg/mL to about 200 mg/mL of the suspension.

12. The injectable bulking agent of claim 9, wherein the extracellular matrix material is porcine small intestine submucosa comprising the at least a portion of the 1.0 ng/mL of the first growth factor in the suspension.

13. The injectable bulking agent of claim 9, where the first growth factor is selected from the group consisting of: FGF1, FGF2, FGF3, FGF4, FGF5, FGF6, FGF7, FGF8, FGF9, FGF10, VEGF A, VEGF B, VEGF C, VEGF D, and VEGF E, PIGF, PDGF, EGF, IFN-alpha, IFN-beta, or IFN-gamma, TGF-alpha, and TGF-beta.

14. The injectable bulking agent of claim 9, wherein the first growth factor is FGF-2.

15. The injectable bulking agent of claim 14, wherein the suspension further comprises a vascular endothelial growth factor.

16. The injectable bulking agent of claim 9, where the suspension further comprises a gelling agent, a biodegradable polymer, a cryoprotecting agent, a surfactant, a tensoactive agent, or a buffering agent.

17. The injectable bulking agent of claim 9, wherein the concentration of particles in the suspension is between about 1 mg/mL and about 100 mg/mL.

18. A method for manufacturing an injectable bulking agent comprising the steps of: (1) combining a comminuted extracellular matrix material material comprising a growth factor with a collagen digestion medium, and (2) maintaining the extracellular matrix material at a pH, temperature and for a duration effective to solubilize a detectable portion of telocollagen in the absence of a proteolytic enzyme.

19. The method of claim 18, where the collagen digestion medium is maintained at a pH of less than about 5.0 at a temperature of less than about 10° C. for a duration of at least about 48 hours; where the liquid vehicle is selected from the group consisting of: a phosphate buffered saline, water, and a physiological solution

20. The method of claim 18, further comprising the steps of: (1) centrifuging the extracellular matrix material to generate a supernatant portion and a pellet portion, isolating the pellet portion, and preparing an injectable bulking agent from the pellet portion, (2) forming a suspension by adding a liquid vehicle to the pellet portion and (3) increasing the pH of the suspension to a physiologically suitable level.
